(19) United States
(12) Patent Application Publication
(10) Pub. No.: US 2011/0014724 A1
(43) Pub. Date: Jan. 20, 2011

(54) METHOD OF DETECTING BIOPRODUCTS USING LOCALIZED SURFACE PLASMON RESONANCE SENSOR OF GOLD NANOPARTICLES

(75) Inventors: Sang Jun Sim, Seoul (KR); Jun Pyo Kim, Suwon-si (KR); Cuong Cao, Suwon (KR)

Correspondence Address: WESTMAN CHAMPLIN & KELLY, P.A., SUITE 1400, 900 SECOND AVENUE SOUTH, MINNEAPOLIS, MN, 55402, US

(73) Assignee: SUNGKYUNKWAN UNIVERSITY FOUNDATION FOR CORPORATE C, Suwon, Gyeonggi-do (KR)

(21) Appl. No.: 12/867,355

(22) PCT Filed: May 26, 2008
(86) PCT No.: PCT/KR2008/002925

§ 371 Date: Sep. 11, 2010

(30) Foreign Application Priority Data
Feb. 13, 2008 (KR) 10-2008-0012921

(51) Int. Cl. G01N 33/553; B05D 1/00; B05D 3/10

(52) U.S. Cl. 436/525; 427/180; 427/337; 977/773; 977/920

(57) ABSTRACT

Disclosed is a method of detecting bioproducts using Localized Surface Plasmon Resonance (LSPR) of gold nanoparticles, which can diagnose bioproducts based on changes in the maximum wavelength occurred by an antigen-antibody reaction after immobilization of the gold nanoparticles onto a glass panel. A sensor using such method exhibits high sensitivity, is low in price, and makes quick diagnosis possible, thereby being applicable to various biological fields associated with environmental contaminants, pathogens and the like, as well as diagnosis of diseases. Further, it provides a technology for manufacturing a sensor having higher sensitivity, low price and quick performance, as compared to conventional methods using SPR.

### TECHNICAL FIELD

The present invention relates to a method of detecting bioproducts using Localized Surface Plasmon Resonance (LSPR) of gold nanoparticles, specifically to a method of detecting bioproducts using a localized surface plasmon resonance sensor of gold nanoparticles which can make diagnosis of bioproducts easy and convenient within a short time by modifying the surface of the gold nanoparticles with ethylene glycol, subjecting it to an antigen-antibody reaction and observing changes in the maximum wavelength.

### BACKGROUND ART

As most of human gene structures have been unveiled owing to the human genome project, researches on the function of human genes and proteomics have drawn attentions of many researchers. Reflecting such tendency, researches on biochips have been diversified, which had been mostly focused on DNA chips, to protein chips, carbohydrate chips, cell chips, and the like, and thus their applications have been extended to various fields such as drug screening, diagnosis of diseases, monitoring of environmental contamination, food stability estimation and the like. Among them, as a method for detection and assay of bio-specimen, there are electrophoresis, mass spectrometry and fluorescence method which have been widely used by using conventional analytical devices. In the above, the two-dimensional electrophoresis has problems such as poor reproducibility, poor separation of alkaline protein and high molecular weight protein, and difficulties in automation. The mass spectrometry has an advantage of being capable of assaying unknown samples; however difficulties in its downsizing and high performance analysis of multiple samples at high speed have been indicated as its disadvantages. In the fluorescence method such as ELISA, every biomaterial should be uniformly labelled with a fluorescent material, causing inconvenience, and the price of fluorescent dyes is very expensive. Therefore, a novel technique which can overcome said disadvantages of electrophoresis, mass spectrometry and fluorescence analytical method, has been in need. As an alternative analytical technique which has been drawing attentions at present, there is a surface plasmon resonance (SPR) method which can recognize the interactions in biomaterials by measuring changes in refractive index thereof.

Surface plasmons are surface electromagnetic waves propagating along the boundary between a thin metal film and a dielectric material, and the surface plasmon is known as the result of quantization of collective charge density oscillation of electrons occurred in the surface of a thin metal film. The excitation of surface plasmons is referred to surface plasmon resonance. The SPR method is an optics-based method which can measure the interactions between molecules and perform real-time measurement of a reaction proceeding, without using a separate labeling material such as fluorescent materials.

Further, localized surface plasmon that is a collective oscillation of electrons bound to metal nanoparticles have currently suggested possible applications significant in the field of nano-optics as well as bioengineering, drawing attentions of many researchers. The metal nanoparticles are characterized in that the intensity or frequency of surface plasmon adsorption bands varies according to the species of materials used. Further, the frequency of the surface plasmon varies according to materials, on which the nanoparticles are placed, and the size, shape and size distribution of the nanoparticles. Moreover, it is applied to sensors by using its characteristic property such that the color of a metal nanoisland is significantly affected by the refractive index of the surrounding materials.

When applied as a sensor, sensing is achieved by binding a single antibody to colloidal Au nanoparticles, and observing interactions between the antibody and the ligand through changes in surface plasmon resonance adsorption peaks, wherein the changes in the surface plasmon resonance adsorption band are proportion to the ligand concentration and associated with interactive kinematics. Such sensing technique has several advantages such that distance and location between metal nanoisland formed on the substrate and periodic particle array (PPA) can be adjusted; nanoparticles can be conveniently functionalized; and sensing can be performed in repetitive and continuous way.

Two types of transmission localized surface plasmon resonance spectroscopy have been developed: one is vacuum evaporation of 2-10 nm Au nanoisland onto a mica or quartz substrate, in which the maximum adsorption band is observed in the range of 570-630 nm; the other is a method using silver nanoparticles having a triangular shape with a height of 50 nm and a base of 100 nm. When they are placed in different solvents, clear changes in the characteristics of localized surface plasmon resonance (LSPR) adsorption band can be observed.

Prostate cancer is one of malignant diseases in men, particularly a tumor rapidly increasing in developed countries. The neoplasm thereof is slow-growing, and radiation therapy or antiandrogen therapy is effective to this cancer, therefore early diagnosis is a significant issue in this disease. In this cancer, a prostate specific antigen (hereinafter, referred as PSA), that is a glycoprotein, specifically a kind of serine protease, is secreted from the epithelial cells of the prostate gland and produced by prostate cancer cells. There are two types of PSA present in blood: a complex PSA which binds with a protease inhibitor, and a free PSA. The complex PSA is a predominant form of PSA in bloodstream and includes a 1-antichemotrypsin complex PSA (hereinafter, referred as PSA-ACT) or a 2-macroglobulin complex PSA, etc. Among them, two forms of PSA, free PSA and PSA-ACT can be determined by immunoassay. Currently, a kit for measuring total PSA, which has been coming into practical use has a problem of showing difference between the that tested values and the actual value.

### DISCLOSURE

### Technical Problem

The object of the present invention is to provide a method for detecting a target material comprising bioproducts by immobilizing receptors to gold nanoparticles modified by LSPR.

The above-mentioned object of the present invention can be achieved according to the present invention described as below.

### Technical Solution

In order to achieve the above object of the present invention, the present invention provides a method of detecting bioproducts characterized by comprising the steps of: (a) immobilizing receptors onto a localized surface plasmon resonance (LSPR) sensor being comprised of gold nanoparticles, the surface of which is modified with an organic adsorbent, and a cover glass where the gold nanoparticles are fixed; (b) flowing a target material on the sensor having immobilized receptors and (c) determining light-scattering spectra by using dark-field microscopy and a resonant Rayleigh scattering micro-spectroscopy system and analyzing mobility of the maximum wavelength.

In the above step (a), it is preferred to immobilize the receptors by using EDC/NHS(1-ethyl-3-(3-dimethylaminopropyl)-carbodiimide hydrochloride/N-hydroxylsuccinimide) solution.

In the above step (c), spectrograph and a CCD camera can be equipped to the resonant Rayleigh scattering micro-spectroscopy system so as to measure the maximum wavelength.

The average particle size of the Au nanoparticles are preferably in the range of 11 nm˜70 nm, and most preferably 30 nm.

The organic adsorbent is a material which can form a self-assembled monolayer on the surface of the Au nanoparticles with minimized non-specific adsorption, and may be, for example, ethylene glycol compounds comprising a thiol group (—SH) and a carboxyl group (—COOH) at the end, or ethylene glycol compounds comprising a thiol group and a hydroxyl group (—OH) at the end. Specifically, as for the organic adsorbent, it is preferred to use a mixed solution of HS(CH₂)₁₁(OCH₂CH₂)₆OCH₂COOH and HS(CH₂)₁₁(OCH₂CH₂)₃OH at a mixing ratio of 1:1 to 1:20 by volume. The most preferred mixing ratio is —COOH:—OH=1:10 (v/v).

The receptors are preferably selected from the group consisting of antibody, DNA, aptamer, substrate for enzyme, amino acid, peptide, lipid, nucleic acid, carbohydrate, cofactor and fragment of antibody (Fab).

According to one embodiment of the present invention, provided is a method of detecting PSA, characterized by comprising the steps of: (a) immobilizing PSA onto a localized surface plasmon resonance (LSPR) sensor comprised of gold nanoparticles, the surface of which is modified with an organic adsorbent, and a cover glass where the gold nanoparticles are immobilized; (b) flowing PSA-ACT complex onto the sensor having the immobilized PSA and (c) determining light-scattering spectra by using dark-field microscopy and a resonant Rayleigh scattering micro-spectroscopy system and analyzing mobility of the maximum wavelength.

The present invention further provides a method of manufacturing a localized surface plasmon resonance (LSPR) sensor for detecting bioproducts, characterized by comprising the steps of: (a) preparing Au nanoparticles; (b) dispersing and immobilizing the Au nanoparticles onto a cover glass and (c) conducting surface treatment of the immobilized Au nanoparticles with an organic adsorbent.

In the step (a), it is preferred to prepare the Au nanoparticles by forming an Au colloidal solution through reduction of a hydrogen tetrachloroaurate solution with a sodium citrate solution.

In the step (c), the organic adsorbent is a material which can form a self-assembled monolayer on the surface of the Au nanoparticles with minimized non-specific adsorption, and may be, for example, ethylene glycol compounds comprising a thiol group (—SH) and a carboxyl group (—COOH) at the end, or ethylene glycol compounds comprising a thiol group and a hydroxyl group (—OH) at the end. Preferably, a mixed solution of HS(CH₂)₁₁(OCH₂CH₂)₆OCH₂COOH and HS(CH₂)₁₁(OCH₂CH₂)₃OH at a mixing ratio of 1:1 to 1:20 can be used, with the most preferred mixing ratio of —COOH:—OH=1:10(v/v).

Hereinafter, the present invention is further described in detail.

The present inventors recognized that detection of bioproducts using LSPR has not been made yet, and thus developed a method for detecting bioproducts such as PSA that is a diagnostic marker of prostate cancer, by using LSPR of Au nanoparticles, as a biochip or biosensor measurement technique.

For manufacturing a LSPR-based sensor according to the present invention, firstly a colloidal solution of the Au nanoparticles is prepared, based on reduction of hydrogen tetrachloroaurate (HAuCl₄) by using sodium citrate. At this time, the size of the Au nanoparticles present in the colloidal solution can be adjusted depending on the amount of the citrate used. After preparation of Au nanoparticles, they are adsorbed onto a transparent glass panel. The Au nanoparticles are modified by ethylene glycol, and the antibody is immobilized by using EDC/NHS [(1-ethyl-3-(3-dimethyl aminopropyl)-carbodiimide hydrochloride)/(N-hydroxylsuccinimide)]. After that, changes in light scattering are measured, confirming detection of a target material.

In the first step, the Au nanoparticles are dispersed and immobilized onto a glass panel as shown in FIG. 1. By taking a picture of a dark field image of the Au nanoparticles immobilized as such, the difference in color depending on the size of particles was observed (FIG. 2). As a result, it was confirmed that the color changed from blue to red. Further, by determining a light scattering spectrum by using a CCD camera, it is found that the maximum wavelength varies depending on the size of nanoparticles (FIG. 2). Like this, upon binding of a bioproduct to the Au nanoparticles, surface morphology of the nanoparticles changes according to the amount of material bound thereto, therefore it is possible to sense bioproducts through such changes in the maximum wavelength.

Then, as a next step, for binding biomolecules to the Au nanoparticles by using EDC/NHS method, ethylene glycol having sulfur and a carboxyl group bound at its end [HS(CH₂)₁₁ (OCH₂CH₂)₆OCH₂COOH] and ethylene glycol having sulfur and a hydroxyl group bound at its end [HS(CH₂)₁₁ (OCH₂CH₂)₃OH] are mixed with the Au nanoparticles so as to modify the surface of the Au nanoparticles. After that, an antibody to a target material is immobilized by using EDC/NHS. After reacting it with a target material, changes in light scattering are measured. When other materials bind to the Au nanoparticles, it is found that the maximum wavelength gradually moves. By measuring such mobility of the maximum wavelength, it is possible to sense bioproducts.

### ADVANTAGEOUS EFFECTS

As it has been described above, the present invention relates to a method of detecting bioproducts using LSPR of gold nanoparticles, which can diagnose bioproducts by measuring changes of the maximum wavelength occurred by an antigen-antibody reaction by using a sensor which is manufactured by immobilizing the gold nanoparticles onto a glass panel and modifying the surface. The sensor is high in sensitivity and low in price, and makes diagnosis possible within a short period, thereby, other than diagnosis of diseases, possibly having various biological applications such as diagnosis of environmental contaminants, pathogens and the like.

### DESCRIPTION OF DRAWINGS

FIG. 1 is a concept view related to a detection method of bioproducts by using localized plasmon resonance of Au nanoparticles.

FIG. 2 is a photo showing a dark field image of Au nanoparticles at 600 magnifications and light scattering spectra according to the size of Au nanoparticles.

FIG. 3 is a view illustrating a schematic structure of a resonant Rayleigh scattering micro-spectroscopy system used in the present invention.

FIG. 4 is a graph representing the changes of the maximum wavelength in the Rayleigh light scattering spectra, when bioproducts are bound to Au nanoparticles, according to the example of the present invention.

FIG. 5 is a graph showing the maximum wavelengths measured before and after surface modification of Au nanoparticles and at the time binding with an antibody and an antigen.

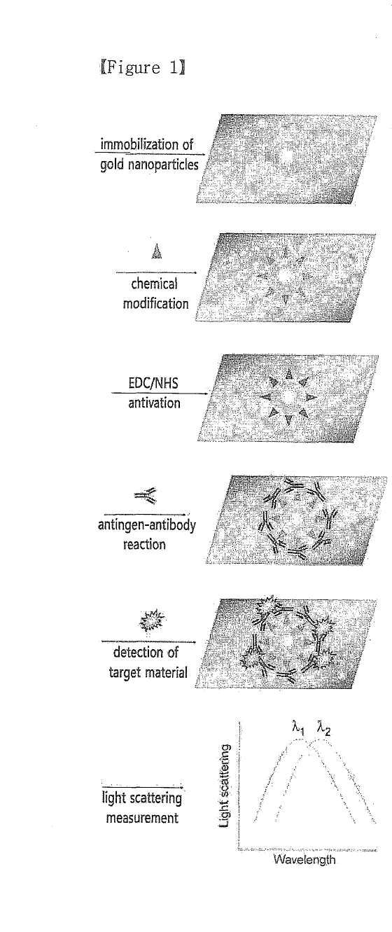

### MODE FOR INVENTION

Hereinafter, for helping to better understand the present invention, preferred examples of the present invention are suggested. These examples are only to illustrate the present invention, by no means limiting the scope of the present invention, and an ordinarily skilled person in the art will clearly understand that various changes or modifications can be made to the examples within the technical scope and spirit of the present invention, wherein such changes or modifications clearly pertain to Claims of the present invention attached to this specification.

### EXAMPLES

### Example 1

Step 1: Preparation of Au Nanoparticles

Au nanoparticles were prepared by reducing a hydrogen tetrachloroaurate (HAuCl₄) solution by using sodium citrate. To a 50 ml erlenmeyer flask, 20 ml of HAuCl₄(1.0 mM) solution was added under heat while stirring, and to the boiling solution, 2 ml of 1% sodium citrate solution was added and stirred rapidly. After that, it was stirred about 30 minutes further, and then cooled at room temperature. As the reducing effect of citrate to gold(III), Au colloid was gradually formed. According to the above described method, prepared were Au nanoparticles in scarlet having an average particle size of about 21 nm.

After forming the colloidal solution, the Au nanoparticles were cultured in a mixed solution of 9 ml of Au solution and 1 ml of a mixed solution of HS-OEG₆—COOH/HS-OEG₃—OH at the mixing ratio of 1:10(v/v).

After mild stirring at room temperature for 6 hours, unreacted parts between the Au nanoparticles and ethylene glycol were separated from the mixed solution by centrifugation (14000 rpm, 15 minutes). Next, the pellets obtained from centrifugation were centrifuged again in 0.1M MES buffer (pH 6.0). The above procedure was repeated 3 times.

Step 2: Immobilization and Modification of the Nanoparticles

A cover glass was put on a RC-30 closed bath chamber, and Au nanoparticle colloid was injected thereinto, for immobilization of the Au nanoparticles onto the cover glass. Then, 1 ml of HS-OEG₆-COOH/HS-OEG₃—OH solution (1:10(v/v), 0.4 mM) was further injected into the chamber and cultured for 24 hours, forming a self-assembled monolayer (SAM) on the surface of the Au nanoparticles.

Step 3: Immobilization of Antibody

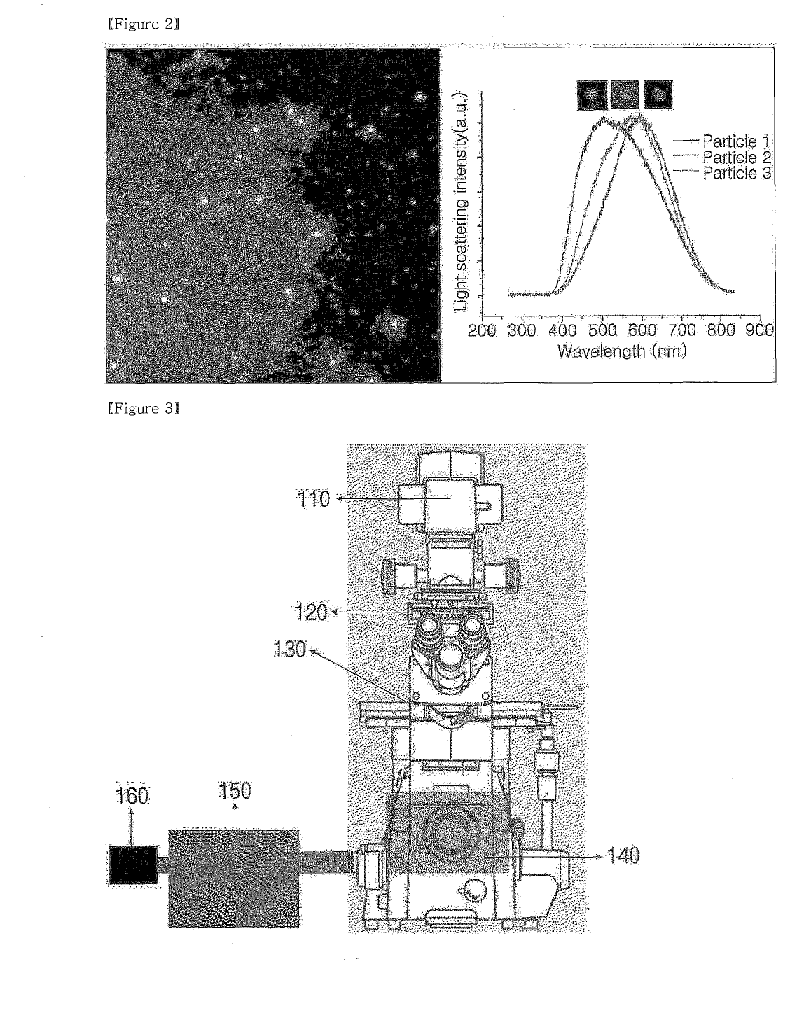

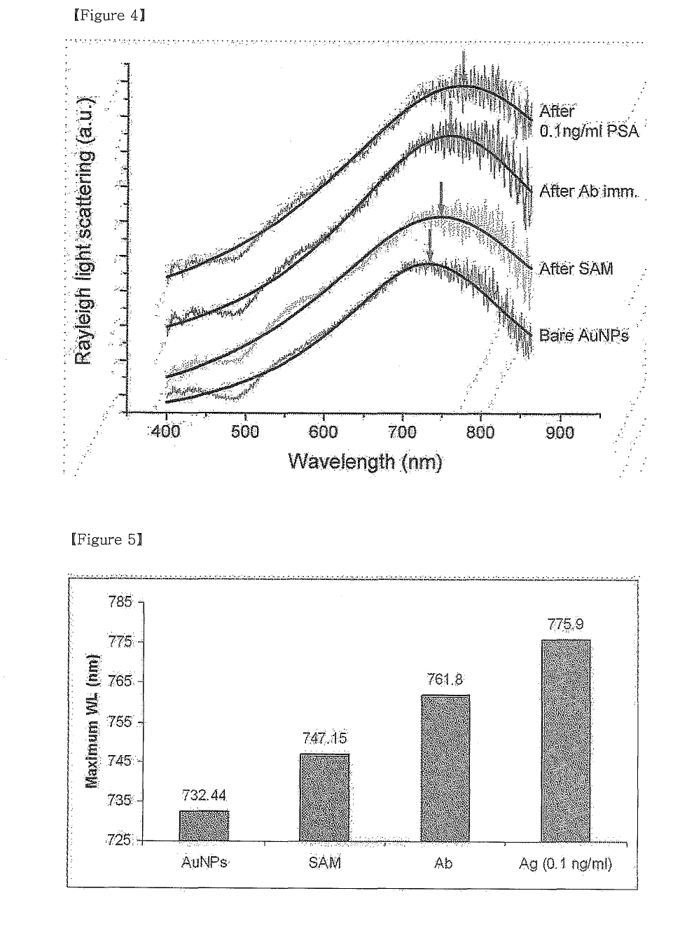

In order to immobilize PAS antibodies to the Au nanoparticle surface, a solution of 0.1 M EDC/NHS(1-ethyl-3-(3-dimethylaminopropyl)-carbodiimide hydrochloride/N-hydroxylsuccinimide) was put into the chamber, activating the carboxyl groups, and then PAS antibodies (10 μg/ml) were injected and then cultured 1 hour. After that, PSA antibodies which were not immobilized were washed out with PBS buffer. The target material, PSA-ACT complex (0.1 ng/ml) was injected into the chamber, and left 1 hour for inducing an antigen-antibody reaction. The changes of wavelength were measured by using dark-field microscopy and charge-coupled device (CCD) camera. Used was a Rayleigh scattering micro-spectroscopy system equipped with an unpolarized light source (110), a dark-field condenser (120), a flow-cell holder (130), a color camera (140), spectrography (150) and a CCD camera (160) as shown in FIG. 3. The maximum wavelengths determined were represented in Table 1 below, and the graphs thereof were shown in FIGS. 4 and 5.

TABLE 1

| | Max. Wavelength (nm) |
|---|---|
| Bare AuNPs | 732.44 |
| After formation of SAM(Self-Assembled | 747.15 |
| Monolayer) | |
| After PSA antibody injection | 761.80 |
| After PSA-ACT complex injection | 775.90 |

The wavelength of the Au nanoparticles immobilized on the glass panel was 732.44 nm, and after modification of the surface of the Au nanoparticles, the wavelength was increased to 747.15 nm. When PSA antibodies were bound to the Au nanoparticles by using EDC/NHS, the wavelength was changed to 761.8 nm. Further, after flowing the target material, PSA-ACT complex, the wavelength was increased to 775.9 nm. It means that mobility of the maximum wavelength was increased to 14.1 nm. As shown above, it is possible to detect a very small amount of PSA-ACT complex such as 100 pg/ml by measuring increase in the maximum wavelength.

1. A method of detecting bioproducts characterized by comprising the steps of:
(a) immobilizing receptors onto a localized surface plasmon resonance (LSPR) sensor comprised of gold nanoparticles, the surface of which is modified with an organic adsorbent, and a cover glass where the gold nanoparticles are fixed;
(b) flowing a target material on the sensor having the immobilized receptors and
(c) determining light-scattering spectra by using dark-field microscopy and a resonant Rayleigh scattering micro-spectroscopy system and analyzing mobility of the maximum wavelength.

2. The method of detecting bioproducts according to claim 1, wherein, in the step (a), the receptors are immobilized by using EDC/NHS(1-ethyl-3-(3-dimethylaminopropyl)-carbodiimide hydrochloride/N-hydroxylsuccinimide) solution.

3. The method of detecting bioproducts according to claim 1, wherein, in the step (c), spectrograph and a CCD camera are equipped to the resonant Rayleigh scattering micro-spectroscopy system so as to measure the maximum wavelength.

4. The method of detecting bioproducts according to claim 1, wherein the average particle size of the Au nanoparticles is in the range of 11 nm to 70 nm.

5. The method of detecting bioproducts according to claim 4, wherein the average particle size of the Au nanoparticles is 30 nm.

6. The method of detecting bioproducts according to claim 1, wherein the organic adsorbent is a material which can form a self-assembled monolayer on the surface of the Au nanoparticles with minimized non-specific adsorption, and comprises ethylene glycol compounds comprising a thiol group (—SH) and a carboxyl group (—COOH) at the end, or ethylene glycol compounds comprising a thiol group and a hydroxyl group (—OH) at the end.

7. The method of detecting bioproducts according to claim 6, wherein the organic adsorbent is a mixed solution of HS(CH₂)₁₁(OCH₂CH₂)₆OCH₂COOH and HS(CH₂)₁₁(OCH₂CH₂)₃OH at a mixing ratio of 1:1 to 1:20.

8. The method of detecting bioproducts according to claim 1, wherein the receptors are selected from the group consisting of antibody, DNA, aptamer, substrate for enzyme, amino acid, peptide, lipid, nucleic acid, carbohydrate, cofactor and Fab.

9. A method of detecting PSA, characterized by comprising the steps of:
(a) immobilizing PSA onto a localized surface plasmon resonance (LSPR) sensor comprised of gold nanoparticles, the surface of which is modified with an organic adsorbent, and a cover glass where the gold nanoparticles are immobilized
(b) flowing PSA-ACT complex onto the sensor having the immobilized PSA and
(c) determining light-scattering spectra by using dark-field microscopy and a resonant Rayleigh scattering micro-spectroscopy system and analyzing mobility of the maximum wavelength.

10. A method of manufacturing a localized surface plasmon resonance (LSPR) sensor for detecting bioproducts, characterized by comprising the steps of:
(a) preparing Au nanoparticles;
(b) dispersing and immobilizing the Au nanoparticles onto a cover glass; and
(c) conducting surface treatment of the immobilized Au nanoparticles with an organic adsorbent.

11. The method of manufacturing a localized surface plasmon resonance (LSPR) sensor for detecting bioproducts according to claim 10, wherein, in the step (a), the Au colloidal solution is formed by reducing a hydrogen tetrachloroaurate solution with a sodium citrate solution

12. The method of manufacturing a localized surface plasmon resonance (LSPR) sensor for detecting bioproducts according to claim 10, wherein the organic adsorbent is a material which can form a self-assembled monolayer on the surface of the Au nanoparticles with minimized non-specific adsorption, and comprises ethylene glycol compounds comprising a thiol group (—SH) and a carboxyl group (—COOH) at the end, or ethylene glycol compounds comprising a thiol group and a hydroxyl group (—OH) at the end.

13. The method of manufacturing a localized surface plasmon resonance (LSPR) sensor for detecting bioproducts according to claim 12, wherein the organic adsorbent is a mixed solution of HS(CH₂)₁₁(OCH₂CH₂)₆OCH₂COOH and HS(CH₂)₁₁(OCH₂CH₂)₃OH at a mixing ratio of 1:1 to 1:20.
